(19) United States
(12) Patent Application Publication
(10) Pub. No.: US 2026/0153499 A1
(43) Pub. Date: Jun. 4, 2026

(54) FISH EMBRYO TEST PLATE AND METHOD OF EVALUATING DEVELOPMENTAL NEUROTOXICITY USING THE SAME

(71) Applicants: KOREA RESEARCH INSTITUTE OF CHEMICAL TECHNOLOGY, Daejeon (KR); National Institute of Environmental Research, Incheon (KR); The Industry & Academic Cooperation in Chungnam National University, Daejeon (KR)

(72) Inventors: Cheol-Hee KIM, Daejeon (KR); Sang Hee LEE, Incheon (KR); Ji Young LEE, Incheon (KR); Jungkwan SEO, Incheon (KR); Sun Kyung SHIN, Gyeonggi-do (KR); Woo-Keun KIM, Daejeon (KR); Sangwoo LEE, Daejeon (KR)

(21) Appl. No.: 19/300,299

(22) Filed: Aug. 14, 2025

(30) Foreign Application Priority Data
Dec. 3, 2024 (KR) 10-2024-0177842

(51) Int. Cl. G01N 33/50; C12M 1/32; C12M 1/34

(52) U.S. Cl. CPC G01N 33/5088; C12M 23/12; C12M 41/46; G01N 33/5014; G01N 2333/4603

(57) ABSTRACT

The present disclosure relates to a plate for evaluating toxicants using fish embryos, a method of screening developmental neurotoxicants using the same, and an apparatus and a kit for evaluating developmental neurotoxicants including the plate. The plate and the screening method of the present disclosure provide a novel test method utilizing characteristics of the swim bladder formation process in fish embryos. This method offers an alternative to animal testing and enables effective toxicity evaluation with a small amount of sample.

### CROSS-REFERENCE TO RELATED APPLICATION

This application claims the benefit of Korean Patent Application No. 10-2024-0177842, filed on Dec. 3, 2024, in the Korean Intellectual Property Office, the entire disclosure of which is incorporated herein by reference for all purposes.

### BACKGROUND

### 1. Field of the Invention

The present disclosure relates to a plate for evaluating toxicants based on a non-animal method using fish embryos, and a corresponding method for evaluating developmental neurotoxicity.

### 2. Description of the Related Art

Conventional evaluation of developmental neurotoxicity has relied on Organization for Economic Cooperation and Development (OECD) Test Guideline (TG) 426: Developmental Neurotoxicity Study, which is based on studies using mammals such as rodents, to evaluate the hazards of chemical substances. OECD TG 426 evaluates the effects related to overall neurological development, including neurological changes in the maternal animal after birth and the behavior, motor activity, learning, and memory of the offspring.

Growing ethical demand for reduced animal testing has necessitated need to introduce alternative test organisms, and validate test methods for evaluating chemical toxicity. This trend has been continuously discussed in the international community. Consequently, various alternative test methods for developmental neurotoxicity evaluation are being actively developed and applied. In the United States, the National Institutes of Health (NIH), under the Department of Health and Human Services, provides a list of alternative test methods accepted by the U.S. government, with reference to guidelines issued by organizations such as the Organization for Economic Co-operation and Development (OECD), the U.S. Food and Drug Administration (FDA), and the U.S. Environmental Protection Agency (EPA). In order to replace mammals, particularly rodents, which are widely used in toxicity testing, alternative test methods using non-animal organisms such as fish embryos are being proposed.

For example, in 2013, the OECD published TG 236: OECD Guidelines for the Testing of Chemicals—Fish Embryo Acute Toxicity (FET) Test, a method for evaluating acute toxicity in fish embryos. The principle of the FET test is as follows:

Zebrafish eggs are exposed to test chemical substances for a period of 96 hours. At the end of the exposure period, the corresponding chemical substances and corresponding concentrations are determined to have acute toxicity if any of the following four observations are made: (i) coagulation of fertilized eggs, (ii) lack of somite formation, (iii) lack of detachment of the tail-bud from the yolk sac, and (iv) lack of heartbeat. LC₅₀ is then calculated based on the results (see [OECD, Test No. “236: FET test.” OECD Guidelines for the Testing of Chemicals, Section 2 (2013): 1-22]).

In humans, the ultimate outcomes of neurodevelopmental abnormalities include not only various postnatal neurological disorders but also prenatal miscarriage and stillbirth at the time of delivery. The first cry of a newborn immediately after birth enables independent pulmonary respiration, and failure to initiate this cry is a major cause of stillbirth. Respiration in animals is maintained through complex regulation by the nervous and muscular systems. The swim bladder of fish is an organ homologous to the lungs of mammals in evolutionary terms, and the formation and function of the swim bladder also develop through intricate interactions between the autonomic nervous and muscular systems during developmental stages. The inventors sought to utilize these complex stages of swim bladder development in fish as an index for evaluating developmental neurotoxicity.

### SUMMARY

One aspect of the disclosure provides a plate for evaluating and screening developmental neurotoxicants using fish embryos.

According to another aspect, there is provided a method of screening developmental neurotoxicity using fish embryos, the method including the plate.

According to still another aspect, there is provided an apparatus for evaluating toxicants using fish embryos, the apparatus including the plate.

According to yet another aspect, there is provided a kit for evaluating developmental neurotoxicity using fish embryos, the kit including the plate.

A plate for evaluating toxicants using fish embryos, according to an embodiment, may include n outer wells; and n inner wells respectively located inside the outer wells, wherein n is an integer of 1≤n≤96, and a height of the inner wells may be less than a height of the outer wells.

The inner wells may be non-permeable.

The material of the plate may be one of polystyrene (PS), polycarbonate (PC), polypropylene (PP), polymethyl methacrylate (PMMA), polyethylene terephthalate (PET), polylactic acid (PLA), polyvinyl chloride, and combinations thereof.

The outer wells and the inner wells of the plate may be filled with a control solution or a test solution, and the liquid is filled to a height which is less than the height of the outer wells but greater than the height of the inner wells.

The height of the inner wells may be about 5 mm to about 21 mm, and a difference (Δh) between the height of the inner wells and the height of the liquid satisfies the relationship of Equation 1.

$$3 mm \leq Liquid height - Inner well height ( Δ h ) \leq 10 mm \qquad [ Equation 1 ]$$

A height ratio of the inner wells to the outer wells may be about 1:3 to about 1:1.2.

A shape of each of the outer wells or the inner wells may be either a prism or a cylinder.

A diameter of the outer wells may be about 10 mm to about 40 mm, and may satisfy a relationship of Equation 2.

$$1.1 \leq Outer well diameter / Inner well diameter \leq 4 \qquad [ Equation 2 ]$$

The fish embryos may be zebrafish embryos.

The plate may be a plate for screening developmental neurotoxicants using fish embryos.

A method, according to an embodiment, may be a method of screening developmental neurotoxicants using fish embryos, the method including: a) providing the plate; b) filling the plate with water or a test solution such that a height of the water or the test solution is greater than a height of inner wells and less than a height of outer wells, and positioning fish embryos in the inner wells; c) when the plate is filled with water in b), introducing a sample into the inner wells of the plate; and d) identifying whether larvae formed from the fish embryos move between the inner wells and the corresponding outer wells.

The fish embryos may be zebrafish embryos.

The fish embryos in b) may be fish embryos from immediately after fertilization to about 48 hours.

d) may be performed 100 to 150 hours after fertilization of the fish embryos.

d) may include counting the number of larvae that failed to move between the inner wells and the corresponding outer wells, and the number of larvae that succeeded in moving between the inner wells and the corresponding outer wells, respectively.

Among the larvae that succeeded in moving between the inner wells and the corresponding outer wells, the larvae may be determined to have succeeded in swim bladder inflation when the swim bladders of the larvae expand into a round, balloon-like shape.

The method of screening may further include e) determining that the sample is a toxicant if the number of larvae that succeeded in swim bladder inflation is 50% or less of the total number of embryos.

An apparatus, according to an embodiment, may be an apparatus for evaluating toxicants using fish embryos, the apparatus including: the plate; and an image reader configured to perform the above-described counting through automated image analysis.

The fish embryos may be zebrafish embryos.

A kit, according to an embodiment, may be a kit for evaluating developmental neurotoxicants using fish embryos, the kit including: the plate, and instructions for using the plate in a method of screening developmental neurotoxicants using fish embryos.

The fish embryos may be zebrafish embryos.

The plate of the present disclosure includes inner wells, thereby enabling the evaluation of swim bladder inflation behavior of fish embryos separately from reduced motility caused by general developmental toxicity. Therefore, the plate of the present disclosure may be used to evaluate the developmental neurotoxicity of chemical substances, and its screening method is about 10 times more sensitive than conventional approaches, enabling accurate analysis with a small sample size.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1 shows a schematic diagram of swim-up behavior and swim bladder inflation behavior of zebrafish.

FIG. 2 shows a measurement of the swim-up behavior of zebrafish using a conventional glass test tube.

FIG. 3 shows an initial schematic diagram of a plate fabricated by attaching inner wells (IWs) to a 12-well plate and a 6-well plate.

FIG. 4 shows a plate for evaluating toxicants using fish embryos. A plate 1 includes a plurality of wells 100. In FIG. 4, the plate 1, as an example, includes 24 wells 100, but the number of wells is not limited.

FIG. 5 is an enlarged perspective view of one of the wells 100 shown in FIG. 4. Each well 100 includes an outer well 110 and an inner well 120.

FIG. 6 is an enlarged cross-sectional view of one of the wells 100 shown in FIG. 4. Dₒ denotes the diameter of the outer well 110, and Di denotes the diameter of the inner well 120. Lo denotes the height of the outer well 110, and Li denotes the height of the inner well 120.

FIG. 7 shows an example embodiment of a plate.

FIG. 8 shows another example embodiment of the plate.

FIG. 9 shows yet another example embodiment of the plate, and the units of the values are mm.

FIG. 10 shows the experimental results of a negative control group and a positive control group performed by using the plate.

FIG. 11 shows a design example of the method of screening for evaluating developmental neurotoxicity.

FIG. 12 shows an example of success or failure in swim bladder inflation of zebrafish embryos.

FIG. 13 shows a CAD drawing of Plate 5 of Example 1-4 and a prototype fabricated using 3D printing technology.

FIG. 14 shows the results of evaluating developmental neurotoxicity of 30 chemical substances using the plate.

### DETAILED DESCRIPTION

Hereinafter, embodiments will be described in detail with reference to the accompanying drawings. However, various alterations and modifications may be made to the embodiments. Here, the embodiments are not construed as limited to the disclosure. The embodiments should be understood to include all changes, equivalents, and replacements within the idea and the technical scope of the disclosure.

The terminology used herein is for the purpose of describing particular embodiments only and is not to be limiting of the embodiments. As used herein, the singular forms “a,” “an,” and “the” are intended to include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

Unless otherwise defined, all terms including technical and scientific terms used herein have the same meaning as commonly understood by one of ordinary skill in the art to which the embodiments belong. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

When describing the embodiments with reference to the accompanying drawings, like reference numerals refer to like components and a repeated description related thereto will be omitted. In the description of embodiments, detailed description of well-known related structures or functions will be omitted when it is deemed that such description will cause ambiguous interpretation of the present disclosure.

In addition, terms such as first, second, A, B, (a), (b), \textcircled{1}, \textcircled{2}, and the like may be used to describe components of the embodiments. These terms are used only for the purpose of discriminating one component from another component, and the nature, the sequences, or the orders of the components are not limited by the terms.

A component, which has the same common function as a component included in any one embodiment, will be described by using the same name in other embodiments. Unless stated otherwise, the configuration described in any one embodiment may be applied to other embodiments, and repeated descriptions will be omitted.

As used in the present disclosure, the term “about” is understood to refer to a range of values that a person skilled in the art would consider equivalent to the stated value in terms of achieving the same function or result. The term “about” refers to ±20% of the stated numerical value, typically ±10%, often ±5%, and in many cases ±2% of the numerical value. In some embodiments, the term “about” refers to the numerical value itself.

All numerical ranges provided throughout the present disclosure are to be understood as including both the upper and lower endpoints of the range, as well as all narrower subranges falling within the stated range, and all such subranges are considered to be specifically and expressly disclosed herein.

As used in the present disclosure, unless explicitly stated otherwise, singular and plural forms such as “a”, “an”, and “˜s” shall be understood to mean “at least one” or “one or more”, and to include the plural forms of the noun.

Hereinafter, the present disclosure will be described in more detail.

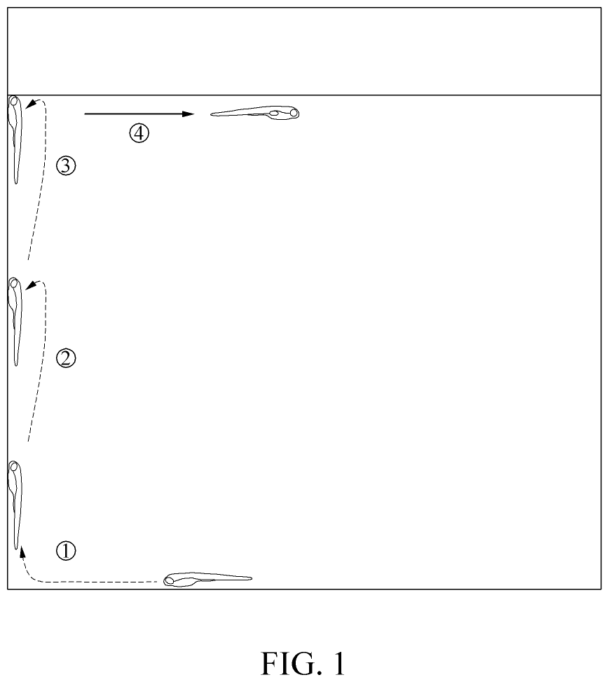

The swim bladder in fish is an organ responsible for regulating buoyancy and is essential for free swimming and survival. Development of the swim bladder in fish requires not only organ formation but also appropriate intake of external air to ensure normal function. For example, in the case of zebrafish embryos, the ability to perform upward-directed movements toward the water surface is necessary to access external air. In particular, normal development of the entire nervous system, including the sensory and motor systems, is required, which includes complex stages such as upward movement in response to gravity, crawling movement along the wall, perception of the position of the water surface, inhalation movement to swallow external air bubbles, sensory regulation to adjust the size of the air bubbles, and ultimately, movement for free swimming (see FIG. 1). The descriptions of each process in FIG. 1 are as follows: \textcircled{1} The zebrafish moves to the waterside and attaches to the wall using the adhesive gland on the jaw. \textcircled{2} The zebrafish swims upward along the wall against gravity and reattaches its jaw to the wall for fixation. \textcircled{3} Process \textcircled{2} is repeated until the zebrafish reaches the water surface, followed by swim bladder inflation. \textcircled{4} By adjusting the inflation of the swim bladder to an appropriate size and achieving neutral buoyancy, the zebrafish begins free swimming and independent movement as an individual organism.

Embryos that fail to develop the swim bladder properly, inhale external air bubbles, or inflate the swim bladder cannot achieve neutral buoyancy, have difficulty feeding through free swimming, and ultimately are unable to survive. Accordingly, the normal swim bladder development process can be utilized as an index for evaluating developmental neurotoxicity and the efficacy of associated neural regulatory substances.

In the case of zebrafish embryos, during normal development, swim bladder inflation occurs between 4 and 5 days post-fertilization, and the embryos are able to survive as individual organisms by controlling buoyancy through various neural functions directly associated with this process, such as dopamine and neuropeptide Y, and engaging in feeding behavior through free swimming (see [Robertson, G. N., et al. “Development of the swimbladder and its innervation in the zebrafish, Danio rerio.” Journal of Morphology 268.11 (2007): 967-985.], and [Smith, Frank M., and Roger P. Croll. “Autonomic control of the swimbladder.” Autonomic Neuroscience 165.1 (2011): 140-148]).

The swim bladder is an organ homologous to the lungs in mammals from an evolutionary perspective. In humans, pulmonary respiration begins with the newborn's first cry immediately after birth, and this process, like in fish, is regulated by proper functioning of the nervous and muscular systems. In this context, abnormalities in neurodevelopment in humans can result not only in various neurological disorders but also in miscarriage or stillbirth. Because the formation and function of the swim bladder also develop through complex interactions between the autonomic nervous and the muscular systems during the developmental stages, the swim bladder can serve as a major indicator for evaluating developmental neurotoxicity. The inventors sought to utilize the swim bladder development in fish as an index for evaluating developmental neurotoxicity based on these characteristics.

In the conventional OECD FET test, zebrafish eggs are exposed to test chemical substances for a period of 96 hours. At the end of the exposure period, the corresponding chemical substances and corresponding concentrations are determined to have acute toxicity if any of the following four observations are made: (i) coagulation of fertilized eggs, (ii) lack of somite formation, (iii) lack of detachment of the tail-bud from the yolk sac, and (iv) lack of heartbeat. LC₅₀ is then calculated based on the results (see [OECD, Test No. “236: Fish embryo acute toxicity (FET) test.” OECD Guidelines for the Testing of Chemicals, Section 2 (2013): 1-22]). In other words, a method of screening developmental neurotoxicants based on the inflation or development of the swim bladder has not been previously established and was newly developed by the present inventors.

The FET test uses several indicators for toxicity evaluation, (i) coagulation of fertilized eggs caused by cytotoxicity, (ii) lack of somite formation and (iii) lack of detachment of the tail-bud from the yolk sac caused by differentiation toxicity, and (iv) lack of heartbeat caused by impaired regulation of cardiac muscle movement. Although abnormalities in one or more of (i) through (iv) may occasionally affect swim bladder development as a secondary effect, this is distinct from the complex regulatory mechanisms of the nervous and muscular systems involved in swim bladder inflation. Accordingly, the present inventors added inner wells as structural components and were able to observe the swim bladder inflation stage of fish embryos through three-dimensional movement, that is, movement in vertical and horizontal directions, which is clearly distinguishable from structural and morphological developmental toxicity caused by early cytotoxicity. In addition, if the embryo succeeds in moving from the inner well to the corresponding outer well (that is, escaping from the inner well), it can be determined that the basic motility of the embryo is not impaired, and allowing the complex process of swim bladder inflation may be distinguished from the simple motility.

The present disclosure relates to a plate for evaluating toxicants using fish embryos, and the plate may include n outer wells; and n inner wells respectively located inside the outer wells, wherein n is an integer of 1≤n≤96, and the height of the inner wells may be less than the height of the outer wells.

Fish refers to an animal among vertebrates that lives in water and breathes through gills. For example, fish may be an OECD standard test species including, but not limited to, zebrafish (Danio rerio), fathead minnow (Pimephales promelas), carp (Cyprinus carpio), medaka (Oryzias latipes), guppy (Poecilia reticulata), bluegill (Lepomis macrochirus), rainbow trout (Oncorhynchus mykiss), three-spined stickleback (Gasterosteus aculeatus), sheepshead minnow (Cyprinodon variegatus), European seabass (Dicentrarchus labrax), or red sea bream (Pagrus major). The fish may be a small fish, including but not limited to, zebrafish (Danio rerio), medaka (Oryzias latipes), Danionella (Danionella cerebrum), or minnow (Phoxinus phoxinus). Preferably, the fish may be a zebrafish. As a small fish species, zebrafish is a vertebrate model like humans and is suitable for large-scale screening studies of pharmaceuticals or chemical substances, as zebrafish can produce hundreds of embryos at once through external fertilization. In addition, zebrafish embryos are transparent, relatively large compared to those of mammals, and develop rapidly, making it easy to observe tissue development and function. According to current international standards, zebrafish embryos up to 5 days post-fertilization are not regarded as animals and can thus be freely used in experiments. OECD TG 236 (FET) also utilizes zebrafish embryos up to 96 hours post-fertilization. Furthermore, the European Union (EU), in Directive 2010/63/EU on the protection of animals used for scientific purposes, only includes larval forms capable of independent feeding within the scope of application.

Fish embryos transform into larvae upon hatching or upon the initiation of exogenous feeding. From this stage, the fish is referred to as a larva until undergoing metamorphosis and becoming a juvenile, and is classified as an adult upon reaching sexual maturity. In the case of zebrafish, hatching typically occurs between 48 and 72 hours after fertilization. Generally, zebrafish embryos are regarded as “larvae” after 72 hours post-fertilization, regardless of whether hatching has occurred. Subsequently, zebrafish embryos undergo metamorphosis and become juveniles around 30 days post-fertilization, and reach sexual maturity after about 3 to 4 months (see [Ali, Shaukat, et al. “Zebrafish embryos and larvae: a new generation of disease models and drug screens.” Birth Defects Research Part C: Embryo Today: Reviews 93.2 (2011): 115-133]).

As used in the present disclosure, the term “fish embryo” broadly refers to the period from immediately after fertilization up to about 72 hours post-fertilization thereafter, and further encompasses the early larval stage up to about 144 hours post-fertilization. In contrast, the term “fish embryo” in a narrow sense refers to the period from immediately after fertilization to about 72 hours post-fertilization, while the term “larva” refers to an individual from about 72 hours to 30 days post-fertilization. Unless otherwise specified, the term “fish embryo” in the present disclosure is used in the broad sense described above.

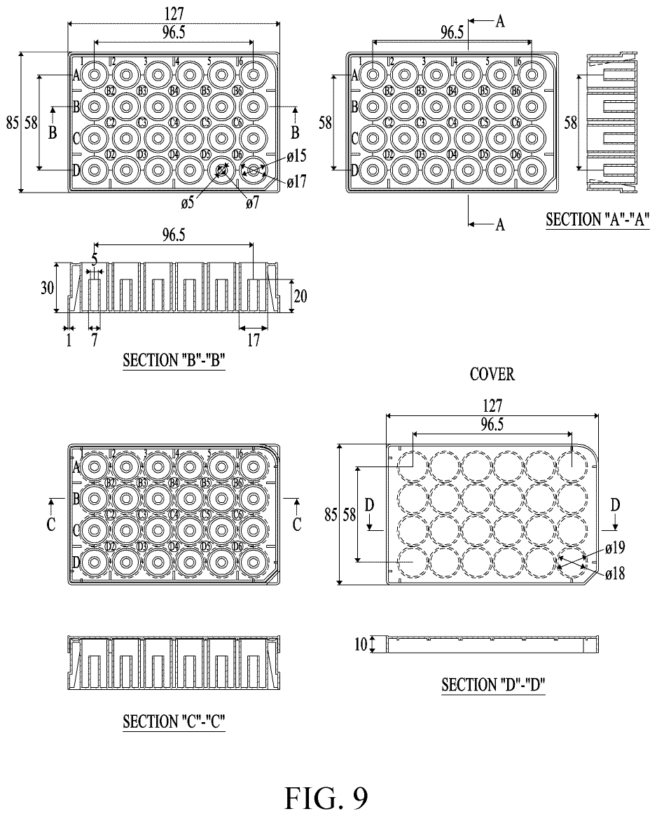

The plate refers to a flat and thin tool mainly used for experiments or research purposes, and includes n wells. n may be an integer of 1 or more, and preferably, n may be an integer of 1≤n≤96, more preferably 6, 12, 24, 48, or 96 but is not limited thereto. The number n may be determined considering the experimenter's convenience or the dilution factor of the drug to be screened. The plate may additionally include a cover that covers the top of the plate (see FIG. 9). In an embodiment, the plate may be a 24-well plate and may further include a cover that covers the top of the plate.

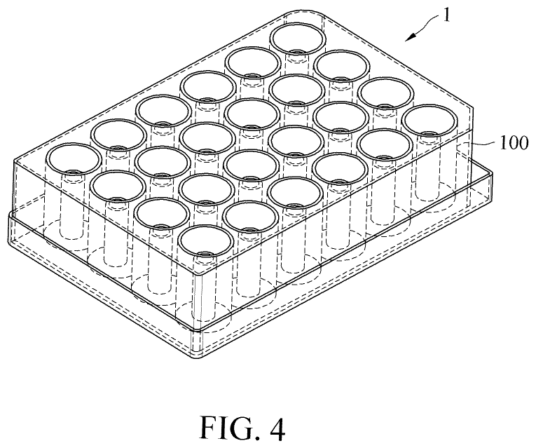

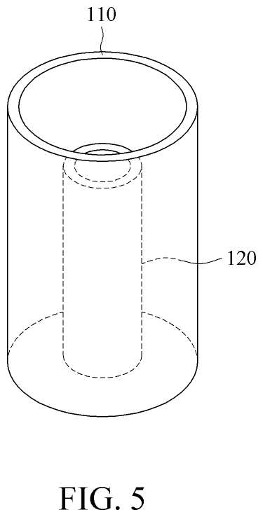

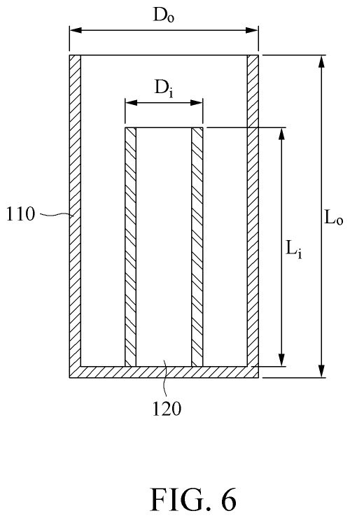

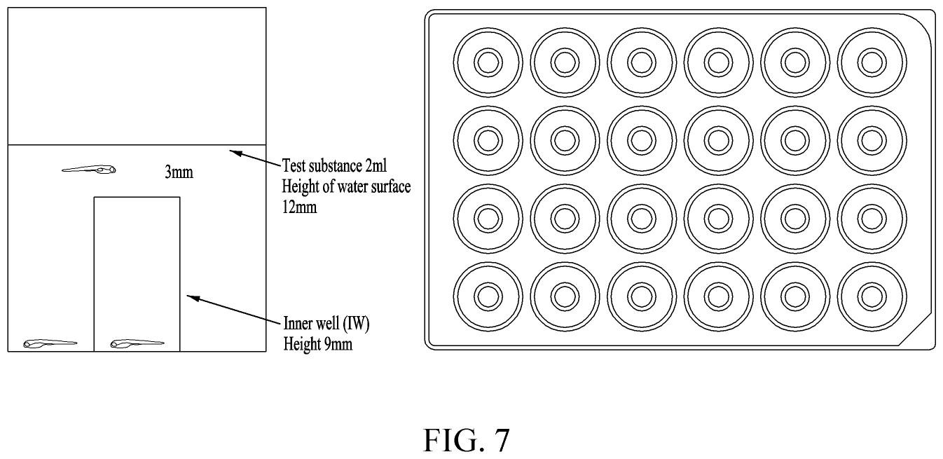

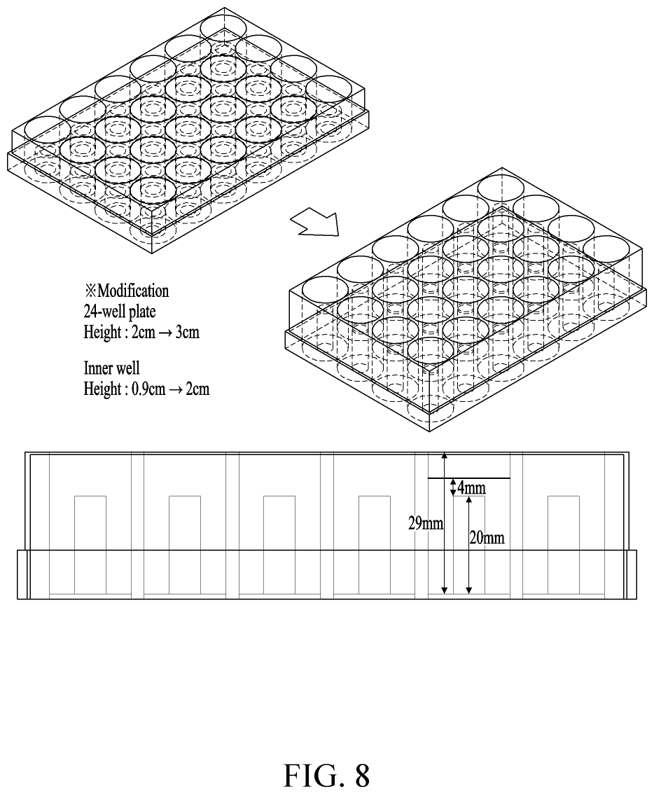

By way of example, and referring to FIGS. 4 to 6, the plate 1 may be a 24-well plate, in which case the plate includes 24 wells 100. The wells 100 may include outer wells 110 and inner wells 120 respectively located inside the outer wells 110. By way of example, and referring to FIG. 7, the plate may be a 24-well plate, in which the height of the inner well may be 9 mm, the height of the water surface may be 12 mm, and 2 mL of a test solution may be added. By way of example, and referring to FIG. 8, the plate may be a 24-well plate, in which the height of the inner well may be 20 mm, the height of the water surface may be 24 mm, and the height of the outer well may be 29 to 30 mm.

The inner wells have a height that is less than that of the outer wells. Because the height (Li) of the inner wells is less than the height (Lo) of the outer wells, when liquid is filled into the wells 100, the surface of the liquid may be positioned above the height of the inner wells while remaining below the height of the outer wells. Through this configuration, it is possible to observe whether the fish embryos move from the inner wells to the outer wells. The inner wells may be permeable, semi-permeable, or non-permeable. Preferably, the inner wells may be non-permeable. The inner wells only need to separate the space of the outer wells from the fish, so it is acceptable for the permeability to be low enough that the fish cannot pass through the inner wells. In other words, as long as the fish cannot move to the outer wells through the wall of the inner wells, there is no limitation on the degree of permeability.

The material of the plate may be one of polystyrene (PS), polycarbonate (PC), polypropylene (PP), polymethyl methacrylate (PMMA), polyethylene terephthalate (PET), polylactic acid (PLA), polyvinyl chloride, and combinations thereof. Preferably, the material of the plate may be PS but is not limited thereto.

The height of the inner wells may be about 5 mm or greater. The height of the inner wells may be about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, or about 21 mm or greater. Preferably, the height of the inner wells may be about 19 mm to about 21 mm but is not limited thereto. When the height of the inner wells is less than about 5 mm, the fish embryos may escape from the inner wells regardless of whether the swim bladder has inflated, so the height of the inner wells may be about 5 mm or greater. The height of the inner wells may be appropriately selected considering the size of the fish embryos.

The height of the outer wells may be about 12 mm or greater. The height of the outer wells may be about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, or about 31 mm or greater. Preferably, the height of the outer wells may be about 28 mm to about 31 mm but is not limited thereto. The height of the outer wells may be appropriately selected considering the height of the inner wells and the size of the fish embryos.

The outer wells and the inner wells of the plate may be filled with a liquid selected from water and a test solution, and the height of the liquid may be less than the height of the outer wells and greater than the height of the inner wells. A difference (Δh) between the height of the inner wells and the height of the liquid may satisfy the relationship of Equation 1.

$$3 mm \leq Liquid height - Inner well height ( Δ h ) \leq 10 mm \qquad [ Equation 1 ]$$

A height ratio of the inner wells and the outer wells may be about 1:3 to about 1:1.2. The height ratio of the inner wells and the outer wells may be about 1:3, about 1:2.9, about 1:2.8, about 1:2.7, about 1:2.6, about 1:2.5, about 1:2.4, about 1:2.3, about 1:2.2, about 1:2.1, about 1:2, about 1:1.9, about 1:1.8, about 1:1.7, about 1:1.6, about 1:1.5, about 1:1.4, about 1:1.3, or about 1:1.2. Preferably, the height ratio of the inner wells and the outer wells may be about 1:1.4 to about 1:1.6 but is not limited thereto.

The shape of each of the outer wells or the inner wells may be either a prism or a cylinder. The shape of the outer wells or the inner wells may be a triangular prism, quadrangular prism, pentagonal prism, hexagonal prism, heptagonal prism, octagonal prism, nonagonal prism, decagonal prism, or cylindrical. Preferably, the shape of the outer wells and the inner wells may be cylindrical but is not limited thereto.

The diameter of the outer wells may be about 10 mm to about 40 mm. Preferably, the diameter of the outer wells may be about 15 mm to about 20 mm but is not limited thereto. The diameter of the inner wells may be about 5 mm to about 35 mm. Preferably, the diameter of the inner wells may be about 5 mm to about 10 mm but is not limited thereto. When the shape of the outer wells or inner wells is a prism, the diameter of a circle inscribed within the polygon that forms the base of the prism can be defined as the diameter. A diameter ratio of the inner wells to the outer wells may be about 1:1.1 to about 1:4. The diameter ratio of the inner wells to the outer wells may be about 1:1.1, about 1:1.2, about 1:1.4, about 1:1.6, about 1:1.8, about 1:2, about 1:2.2, about 1:2.4, about 1:2.6, about 1:2.8, about 1:3, about 1:3.2, about 1:3.4, about 1:3.6, about 1:3.8, or about 1:4. Preferably, the diameter ratio of the inner wells and the outer wells may be about 1:2.4 to about 1:3.2 but is not limited thereto. The diameter of the outer wells and the diameter of the inner wells may satisfy the relationship of Equation 2.

$$1.1 \leq Outer well diameter / Inner well diameter \leq 4 \qquad [ Equation 2 ]$$

The plate may be additionally filled with a liquid selected from water or a test solution. The water or the test solution may be filled to a level greater than the height of the inner wells and less than the height of the outer wells. The plate may be filled with egg water instead of water. The test solution may include a chemical substance or sample to be tested, or may be a solution in which the chemical substance or sample is dissolved. The test solution may be a solution corresponding to a negative control or a positive control. The plate may accommodate fish embryos in the inner wells. The fish and fish embryos are as described above.

The plate may be a plate for screening developmental neurotoxicants using fish embryos. The screening of developmental neurotoxicants refers to the evaluation of the effects of chemical substances, drugs, or samples on the development of the nervous system. As described above, swim bladder inflation or development in fish embryos is closely related to neurodevelopment. Therefore, by placing fish embryos in the inner wells of a plate and treating them with a test solution or sample to evaluate whether swim bladder inflation or development occurs, developmental neurotoxicants can be screened.

The present disclosure relates to a method of screening developmental neurotoxicants using fish embryos, and the method may include: a) providing the plate; b) filling the plate with water or a test solution such that the height of the water or the test solution is greater than the height of inner wells and less than the height of outer wells, and positioning fish embryos in the inner wells; c) when the plate is filled with water in b), introducing a sample into the inner wells of the plate; and d) identifying whether larvae formed from the fish embryos move between the inner wells and the corresponding outer wells.

The fish embryo may be an OECD standard test species embryo including, but not limited to, zebrafish (Danio rerio), fathead minnow (Pimephales promelas), carp (Cyprinus carpio), medaka (Oryzias latipes), guppy (Poecilia reticulata), bluegill (Lepomis macrochirus), rainbow trout (Oncorhynchus mykiss), three-spined stickleback (Gasterosteus aculeatus), sheepshead minnow (Cyprinodon variegatus), European seabass (Dicentrarchus labrax), or red sea bream (Pagrus major). The fish embryo may be an embryo of a small fish, including but not limited to, zebrafish (Danio rerio), medaka (Oryzias latipes), Danionella (Danionella cerebrum), or minnow (Phoxinus phoxinus). Preferably, the fish embryos may be zebrafish embryos. As described above, zebrafish is a vertebrate model like humans and is suitable for large-scale screening studies of pharmaceuticals or chemical substances, as zebrafish can produce hundreds of embryos at once through external fertilization. In addition, zebrafish embryos are transparent, relatively large compared to those of mammals, and develop rapidly, making it easy to observe tissue development and function.

The filling of the plate in b) may involve filling the plate with water or a test solution such that the height of the water or the test solution is greater than the height of inner wells and less than the height of outer wells, and positioning fish embryos in the inner wells. The water may be egg water. For example, the egg water may be prepared by dissolving 6 g of sea salt in 1 L of triple-distilled water to make a concentrated solution, and then diluting the concentrated solution at a ratio of 1:100 to obtain a final concentration of 60 μg/mL. The test solution may include a chemical substance or sample to be tested, or may be a solution in which the chemical substance or sample is dissolved. The test solution may be a solution corresponding to a negative control or a positive control.

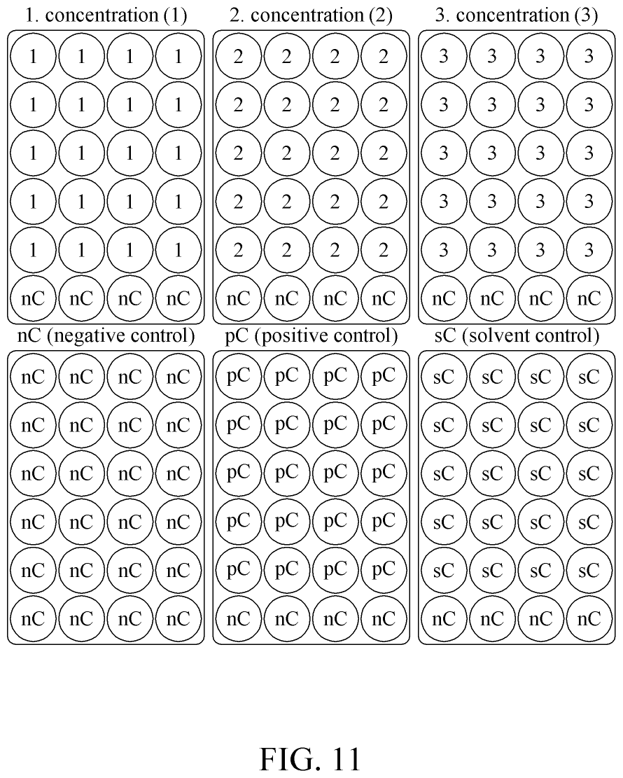

The introducing of a sample in c) may involve introducing a sample, which is to be evaluated for developmental neurotoxicity, into each well of the plate at different concentrations. For example, as shown in FIG. 11, the sample may be introduced into each well (the inner well, or both the inner and outer wells) at three different concentrations along with a solvent control, a negative control, and a positive control. In FIG. 11, 1 to 3 represent experimental groups corresponding to three different concentration conditions of a specific drug, nC represents a negative control (e.g., egg water), pC represents a positive control (e.g., 40 mg/L tricaine), and sC represents a solvent control. For example, the sample may be introduced by varying the concentration to about 25 mg/L, about 12.5 mg/L, and about 6.25 mg/L, by varying the concentration to about 100 mg/L, about 50 mg/L, about 25 mg/L, and about 12.5 mg/L, or by varying the concentration to about 50 mg/L, about 25 mg/L, about 12.5 mg/L, and about 6.25 mg/L. For example, the sample may be introduced at dilution levels of about 2-fold, about 3-fold, about 4-fold, about 10-fold, or about 100-fold. In the introducing of the sample, the method of introducing the sample is not particularly limited, and any method commonly used in the relevant technical field may be used, taking into consideration the chemical characteristics of the sample, the expected toxicity range, and the like.

In b) and c), the water may be filled to a level higher than the height of the inner wells in the plate, fish embryos may be positioned in the inner wells, and the sample may then be introduced into the inner wells of the plate. Here, the sample may be a liquid sample or a solid sample such as powder. Alternatively, in b) and c), the test solution may be filled to a level higher than the height of the inner wells of the plate, and the fish embryos may be positioned in the inner wells. The test solution may include a chemical substance or sample to be tested, or may be a solution in which the chemical substance or sample is dissolved. The test solution may be a solution corresponding to a negative control or a positive control. That is, in b) and c), water may be first filled in the plate and then the sample may be added to the plate, or a test solution in which the sample is dissolved in water may be directly filled in the plate.

d) may include counting the number of larvae that failed to move between the inner wells and the corresponding outer wells, and the number of larvae that succeeded in moving between the inner wells and the corresponding outer wells, respectively. In an embodiment, among the larvae that succeeded in moving between the inner wells and the corresponding outer wells, the larvae may be determined to have succeeded in swim bladder inflation when the swim bladders of the larvae are expanded in a round, balloon-like shape.

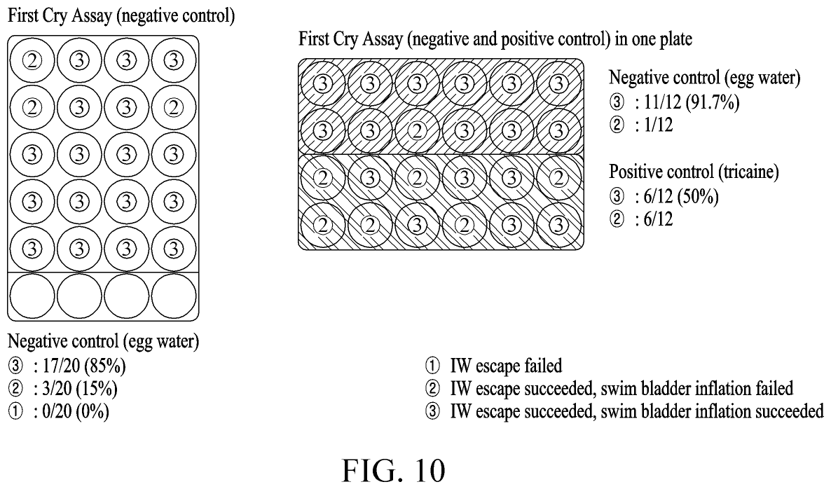

Alternatively, the method of screening may further include quantifying by respectively counting (i) the number of fish embryos that failed to move from the inner wells to the outer wells, (ii) the number of fish embryos that succeeded in moving from the inner wells to the outer wells but failed in swim bladder inflation, and (iii) the number of fish embryos that succeeded in both the movement from the inner wells to the outer wells and the swim bladder inflation in d). Instead of the number of fish embryos, the number of wells in which the fish embryos are located may be counted. One fish embryo may be located in one well, in which case the number of counted wells may be the same as the number of fish embryos but is not limited thereto. By way of example, and referring to FIG. 10, “(1” indicates failure to move from the inner well to the outer well, “(2)” indicates successful movement from the inner well to the outer well but failure in swim bladder inflation, and “(3)” indicates successful movement from the inner well to the outer well as well as successful swim bladder inflation. The left diagram in FIG. 10 shows the result of an experiment in which egg water was used as the negative control. The right diagram in FIG. 10 shows the result of an experiment in which egg water was used as the negative control and tricaine was used as the positive control.

The method of screening may further include calculating an EC₅₀ value based on the above-mentioned quantitative data. For example, when calculating the EC₅₀ (half maximal effective concentration), if the “effect” is defined as “failure of swim bladder formation”, the EC₅₀ value refers to a case where 50% of the individuals have failed to form a swim bladder. The EC₅₀ value may be calculated from the total number of individuals used in the test for each concentration and the sum of (i) the number of fish embryos that failed to move from the inner wells to the outer wells and (ii) the number of fish embryos that succeeded in moving from the inner wells to the outer wells but failed in swim bladder inflation by using a Probit analysis method, which is one of the regression analysis methods, with the statistical program SPSS (IBM). However, the method of obtaining the EC₅₀ value is not particularly limited and any method commonly used in the relevant technical field may be employed.

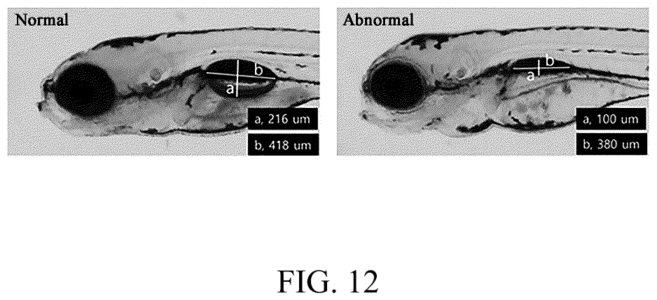

The evaluation of the swim bladder formation of the fish embryos may be performed by microscopic observation to determine the success or failure of swim bladder inflation. The success may refer to a case where the swim bladder of the fish embryo is inflated into a round, balloon-like shape, and the failure may refer to a case where the swim bladder of the fish embryo is flattened (see FIG. 12). The term “successful swim bladder inflation” may be used interchangeably with “normal swim bladder inflation”, and the term “failed swim bladder inflation” may be used interchangeably with “poor swim bladder inflation” or “abnormal swim bladder inflation”.

The microscopic observation may be performed using a stereoscopic microscope but is not limited thereto. Any microscope that can observe swim bladder inflation may be used without limitation. In addition, d) may be performed by automated device observation. Examples of the automated devices may include DanioVision (Noldus), Zebralab (Viewpoint), and the like.

In the case where the swim bladder is inflated into a round, balloon-like shape, when the minor axis (vertical length) of the swim bladder is denoted as “a” and the major axis (horizontal length) is denoted as “b”, it may refer to a case where the value of a/b is about 0.5 or more (see the left diagram in FIG. 12). In an embodiment, when the fish embryos are zebrafish embryos, the value of a may be about 213±5 μm and the value of b may be about 385±11 μm. The size of fish embryos may vary, even within the same species, and a person skilled in the art can readily determine whether the swim bladder of the fish embryo is inflated into a round, balloon-like shape, that is, whether inflation is successful. Therefore, the success of swim bladder inflation is not limited to the above-presented numerical ranges.

In the case where the swim bladder is in a flattened shape, when the minor axis (vertical length) is denoted as “a” and the major axis (horizontal length) is denoted as “b”, it may refer to a case where the value of a/b is less than about 0.5 (see the right diagram in FIG. 12). In an embodiment, when the fish embryos are zebrafish embryos, the value of a may be about 83±7 μm and the value of b may be about 344±18 μm. The size of fish embryos may vary, even within the same species, and a person skilled in the art can readily determine whether the swim bladder of the fish embryo is in a flattened shape, that is, whether inflation has failed. Therefore, the failure of swim bladder inflation is not limited to the above-presented numerical ranges. In an embodiment, the a/b ratio may be about 0.5 or greater when swim bladder inflation is normal (i.e., successful), whereas the a/b ratio may be less than about 0.5 when swim bladder inflation is abnormal (i.e., failed).

The fish embryos in b) may be fish embryos from immediately after fertilization to about 48 hours. In an embodiment, the fish embryos in b) may be fish embryos at about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 49 hours, about 50 hours, about 51 hours, about 52 hours, about 53 hours, about 54 hours, about 55 hours, about 56 hours, about 57 hours, or about 58 hours after fertilization.

d) may be performed 100 hours to 150 hours after fertilization of the fish embryos. d) may be performed about 100 hours, about 101 hours, about 102 hours, about 103 hours, about 104 hours, about 105 hours, about 106 hours, about 107 hours, about 108 hours, about 109 hours, about 110 hours, about 111 hours, about 112 hours, about 113 hours, about 114 hours, about 115 hours, about 116 hours, about 117 hours, about 118 hours, about 119 hours, about 120 hours, about 121 hours, about 122 hours, about 123 hours, about 124 hours, about 125 hours, about 126 hours, about 127 hours, about 128 hours, about 129 hours, about 130 hours, about 131 hours, about 132 hours, about 133 hours, about 134 hours, about 135 hours, about 136 hours, about 137 hours, about 138 hours, about 139 hours, about 140 hours, about 141 hours, about 142 hours, about 143 hours, and about 144 hours after fertilization of the fish embryos. d) may be performed about 36 hours after c), which involves introducing of the test substance.

Sealing the plate may further be included after c). If a cover for the plate is present, the sealing of the plate may involve covering the plate with the cover. The sealing of the plate is intended to prevent evaporation of the water or the test solution, and is not particularly limited.

The present disclosure may be an apparatus for evaluating toxicants using fish embryos, the apparatus including: the plate; and an image reader configured to perform counting the number of larvae that failed to move between the inner wells and the corresponding outer wells, and the number of larvae that succeeded in moving between the inner wells and the corresponding outer wells through automated image analysis.

The plate and the fish embryos are as described above.

The present disclosure may be a kit for evaluating developmental neurotoxicants using fish embryos, the kit including: the plate and instructions for using the plate in a method of screening developmental neurotoxicants using fish embryos.

The plate and the fish embryos are as described above.

The instructions may relate to a method of using the plate in a method of screening developmental neurotoxicants using fish embryos. The instructions may include a method of using the kit, storage and handling, the functions of each component of the kit, and a method of interpreting the results. The instructions may also include descriptions of each procedure of the above-described screening method.

The present disclosure is described in more detail in the examples below.

### Example 1: Preparation of a Plate for Evaluating Swim Bladder Formation and Inflation in Zebrafish

### Example 1-1: Preparation and Evaluation of Plate 1

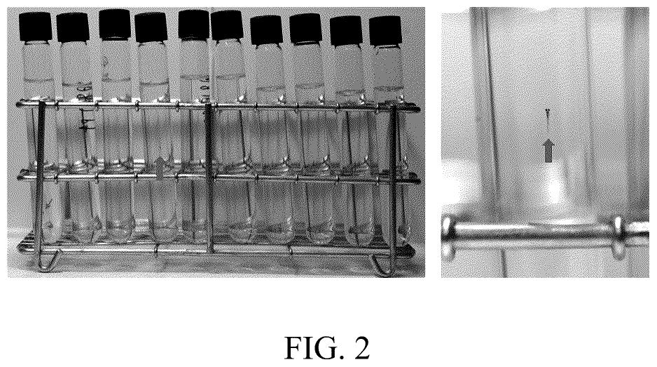

As an initial model for observing swim-up behavior of zebrafish, a method of arranging multiple test tubes as shown in FIG. 2 was used. By using multiple test tubes, multiple embryos can be tested simultaneously, and as a result, the embryos can be treated with various test substances to conduct the experiment.

### Example 1-2: Preparation and Evaluation of Plates 2 and 3

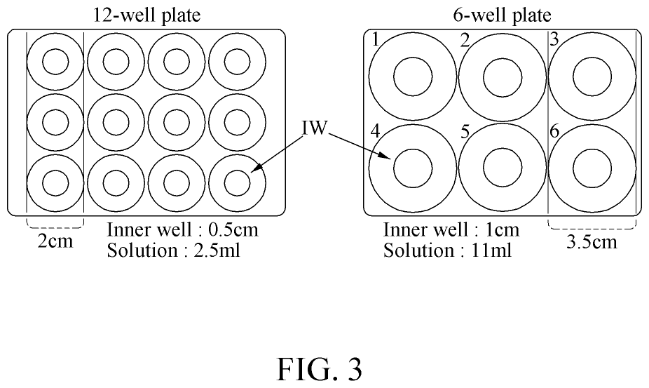

Plates 2 and 3 were prepared for evaluating swim bladder formation and inflation in zebrafish (see FIG. 3). Plate 2 was prepared by cutting 2 mL tubes (Axygen, Microtube Clear 2.0 mL, Cat. No. 27-00619-01, Model: MCT-200-C) into cylindrical shapes with a height of 0.5 cm and attaching the cut tubes to a 12-well plate (SPL Life Science, Korea, Cat. No. 30012). Plate 3 was prepared by cutting 15 mL tubes (SPL Life Science, Korea, Cat. No. 50015) into cylindrical shapes with a height of 1 cm and attaching the cut tubes to a 6-well plate (SPL Life Science, Korea, Cat. No. 30006). The volumes of test substances required to conduct the experiment using Plates 2 and 3 were 2.5 mL and 11 mL, respectively.

In the case of Plate 2, there was an issue in that the inner wells were too low, allowing embryos to escape without performing swim-up behavior. In the case of Plate 3, a limitation was that only six zebrafish embryos could be tested per plate, resulting in excessive consumption of test substances.

### Example 1-3: Preparation and Evaluation of Plate 4

Plate 4 was prepared by attaching inner wells each with a height of 9 mm to each well of a 24-well plate (SPL Life Science, Korea, Cat. No. 30024), thereby creating internal spaces large enough to accommodate a single zebrafish embryo for use in the experiment (see FIG. 7). The 24-well plate is arranged in a 4×6 format. Each well includes an inner well concentric with the outer well. The structure is characterized by the presence of the inner well, which is used to identify the effect of reduced motility in zebrafish, and the outer well, which serves as a waterside space where swim bladder inflation behavior occurs.

If a zebrafish embryo at five days after fertilization fails to pass through the inner well at a specific drug and concentration, this indicates that the embryo was hindered at the swim-up behavior stage, which occurs prior to swim bladder inflation and involves the embryo moving toward the air layer at the waterside. In such cases, it can be determined that swim bladder inflation behavior cannot be evaluated at the corresponding drug and concentration. Conversely, if a zebrafish embryo at five days after fertilization passes through the inner well but exhibits abnormal swim bladder inflation behavior at a specific drug and concentration (e.g., if the embryo fails to swim properly, sinks to the bottom, or lies on its side, such behavior is regarded as an abnormal swim bladder inflation behavior), the effect on swim bladder inflation behavior may be accurately identified independently of impaired motility.

In summary, screening for developmental neurotoxicants using this plate enables identification of swim bladder inflation-related developmental neurotoxicity under various concentration conditions, while excluding motility-related toxicity caused by a specific drug. The experimental results using this plate are described in detail in Example 2.

### Example 1-4: Preparation and Evaluation of Plate 5

In the case of Plate 4, the following issues were identified. First, the height of the inner wells (9 mm) was too low to serve as a threshold for distinguishing motility, as embryos could escape from the inner wells in response to stimulation (escape response), regardless of swim-up behavior. Second, the low height of the inner wells also posed an issue in that even slight vibrations during the experiment could cause embryos to exit the inner wells.

To address these issues, the height of the inner wells was increased from 9 mm to 20 mm, and the height of the outer wells was adjusted to 30 mm so that all of the inner wells could be submerged below the water surface. The adjusted height (20 mm) of the inner wells was appropriate for evaluating motility, as the embryos could not reach the top of the inner wells from the bottom with a single thrusting bout, thereby inducing swim-up behavior. Additionally, escape of embryos from the inner wells due to vibration was significantly reduced.

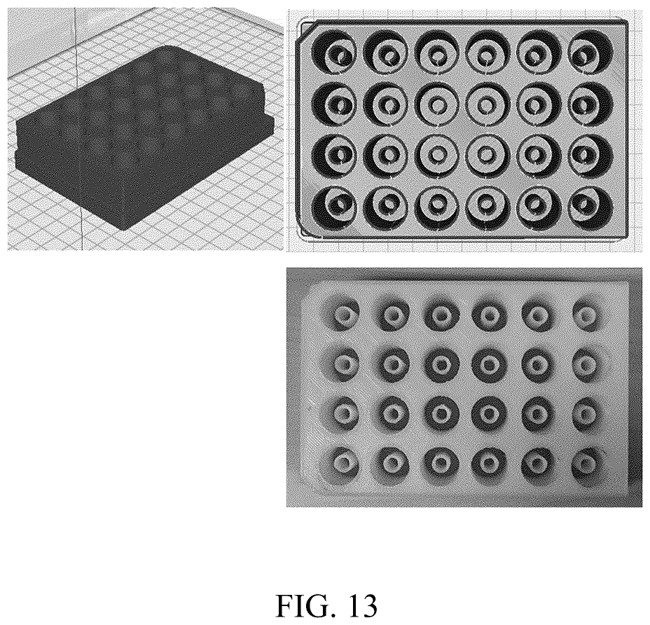

To verify the reproducibility of the experiment using Plate 5, a prototype was fabricated using PLA material via 3D printing technology, and experiments were conducted with zebrafish embryos treated with egg water as a negative control and tricaine (MS-222) as a positive control (see FIG. 13). As a result, Plate 5 yielded the same results as Plate 4 in both the negative and positive controls, while simultaneously reducing embryo escape caused by experimenter error, thereby improving ease of control by the experimenter.

### Example 2: Evaluation of Developmental Neurotoxicity in Zebrafish Using Plate 4

### Example 2-1: Preliminary Experiment for Positive Control

First, before conducting the developmental neurotoxicity evaluation of zebrafish, a preliminary experiment was performed using tricaine (MS-222), which is an anesthetic, as a positive control. Four Plate 4s were used, with each plate treated with tricaine at 80 mg/L, 40 mg/L, and 20 mg/L, and with egg water as a negative control. For example, the egg water may be prepared by dissolving 6 g of sea salt (Instant Ocean, Aquarium Systems) in 1 L of triple-distilled water to make a concentrated solution, and then diluting the concentrated solution at a ratio of 1:100 to obtain a final concentration of 60 μg/mL.

The results are shown in Table 1. In Table 1, “\textcircled{1}” indicates failure to move from the inner well to the outer well, “\textcircled{2}” indicates successful movement from the inner well to the outer well but failure in swim bladder inflation, and “\textcircled{3}” indicates successful movement from the inner well to the outer well as well as successful swim bladder inflation. The experimental results indicated that using tricaine at 40 mg/L is appropriate as a positive control.

TABLE 1

| | | | Total | | | |
|---|---|---|---|---|---|---|
| | | | Number | Number of Wells (Percentage) | | |
| Substance | Solvent | Concentration | of Wells | \textcircled{1} | \textcircled{2} | \textcircled{3} |
| Tricaine | X | 80 mg/L | 20 | 13 (65.0%) | 7 (35.0%) | 0 (0.0%) |
| (MS-222) | | 40 mg/L | 20 | 9 (45.0%) | 11 (55.0%) | 0 (0.0%) |
| | | 20 mg/L | 20 | 0 (0.0%) | 1 (5.0%) | 19 (95.0%) |
| Negative | X | X | 24 | 0 (0.0%) | 3 (12.5%) | 18 (87.5%) |
| Control | | | | | | |
| Internal Plate | X | X | 12 | 0 (0.0%) | 0 (0.0%) | 12 (100.0%) |
| Control | | | | | | |

### Example 2-2: Swim Bladder Development Effect Alternative Test Method (First Cry Assay)—Evaluation of Developmental Neurotoxicity of 30 Chemical Substances Using Plate 4

Zebrafish develop rapidly, with most tissues already formed and functional by one day after fertilization. In addition, behavioral changes due to short-term exposure to chemical substances can be observed in zebrafish embryos (see [Kim, Seong Soon, et al. “Neurochemical effects of 4-(2Chloro-4-fluorobenzyl)-3-(2-thienyl)-1, 2, 4-oxadiazol-5 (4H)-one in the pentylenetetrazole (PTZ)-induced epileptic seizure zebrafish model.” International journal of molecular sciences 22.3 (2021): 1285.]). To exclude secondary effects such as initial cytotoxicity caused by test substances and evaluate only the neurospecific effects, normal embryos with no developmental abnormalities at 24 or 48 hours after fertilization were selected and placed inside each inner well of Plate 4. Next, 4 mL of a test solution was added to each well, and the water level was about 24 mm. Finally, on the fifth day after fertilization (three days after exposure to the test substance), swim bladder inflation was evaluated.

The evaluation of swim bladder inflation was classified and quantified based on the following criteria: \textcircled{1} failure to move from the inner well to the outer well, \textcircled{2} success in moving from the inner well to the outer well but failure in swim bladder inflation, and \textcircled{3} success in both moving from the inner well to the outer well and swim bladder inflation.

Zebrafish at five days after fertilization instinctively move to the waterside environment and perform swim bladder inflation behavior. Therefore, until zebrafish achieve neutral buoyancy via swim bladder inflation, they do not move from the outer well to the inner well and continue swim-up behavior. In controlled experiments, an embryo located in the inner well after completing swim bladder inflation is considered to have returned to the inner well after ingesting an air bubble at the water surface of the outer well, and this is not regarded as a motility impairment. Failure of swim bladder inflation in an embryo located in the inner well is considered a motility impairment. Failure of swim bladder inflation in an embryo located in the outer well is considered an impairment of swim bladder inflation.

The numbers of embryos corresponding to criteria \textcircled{1}, \textcircled{2}, and \textcircled{3} for each of the 30 chemical substances are as follows. The total number of embryos used in the concentration-dependent tests and the sum of the numbers of fish embryos corresponding to \textcircled{1} and \textcircled{2} were used to calculate an EC₅₀ value using a Probit analysis method, which is one of the regression analysis methods, with the statistical program SPSS (IBM). Here, the EC₅₀ value refers to the concentration at which 50% of the embryos fail to form a swim bladder.

(1) 4,4′-Oxydianiline
First cry assay

| Exposure concentration (mg/L) | \textcircled{1} | \textcircled{2} | \textcircled{3} | EC₅₀ (mg/L) |
|---|---|---|---|---|
| 25 | 0 | 12 | 8 | 21.198 |
| 12.5 | 0 | 4 | 16 | |
| 6.25 | 0 | 0 | 20 | |
| | — | — | — | |
| 0 | 0 | 2 | 22 | |

(2) 1,3-Benzenediamine
First cry assay

| Exposure concentration (mg/L) | \textcircled{1} | \textcircled{2} | \textcircled{3} | EC₅₀ (mg/L) |
|---|---|---|---|---|
| 25 | 4 | 15 | 1 | 11.331 |
| 12.5 | 1 | 6 | 13 | |
| 6.25 | 0 | 6 | 14 | |
| | — | — | — | |
| 0 | 0 | 0 | 24 | |

(3) N-Methyl-2-pyrrolidone
First cry assay

| Exposure concentration (mg/L) | \textcircled{1} | \textcircled{2} | \textcircled{3} | EC₅₀ (mg/L) |
|---|---|---|---|---|
| 100 | 0 | 11 | 9 | 95.053 |
| 50 | 0 | 3 | 17 | |
| 25 | 0 | 1 | 19 | |
| | — | — | — | |
| 0 | 0 | 3 | 21 | |

(4) 4-Methylbenzenamine
First cry assay

| Exposure concentration (mg/L) | \textcircled{1} | \textcircled{2} | \textcircled{3} | EC₅₀ (mg/L) |
|---|---|---|---|---|
| 100 | 0 | 20 | 0 | 53.572 |
| 50 | 0 | 6 | 14 | |
| 25 | 0 | 0 | 20 | |
| | — | — | — | |
| 0 | 0 | 0 | 24 | |

(5) 4,4′-Diaminobiphenyl methane
First cry assay

| Exposure concentration (mg/L) | \textcircled{1} | \textcircled{2} | \textcircled{3} | EC₅₀ (mg/L) |
|---|---|---|---|---|
| 25 | 0 | 11 | 9 | 26.133 |
| 12.5 | 0 | 1 | 19 | |
| 6.26 | 0 | 1 | 19 | |
| | — | — | — | |
| 0 | 0 | 0 | 24 | |

(6) 1,4-Benzenediamine
First cry assay

| Exposure concentration (mg/L) | \textcircled{1} | \textcircled{2} | \textcircled{3} | EC₅₀ (mg/L) |
|---|---|---|---|---|
| 25 | 18 | 2 | 0 | 4.023 |
| 12.5 | 12 | 8 | 0 | |
| 6.25 | 4 | 12 | 4 | |
| | — | — | — | |
| 0 | 0 | 0 | 24 | |

(7) Triethylene glycol dimethyl ether
First cry assay

| Exposure concentration (mg/L) | \textcircled{1} | \textcircled{2} | \textcircled{3} | EC₅₀ (mg/L) |
|---|---|---|---|---|
| 50 | 0 | 1 | 19 | >50 |
| 25 | 0 | 0 | 20 | |
| 12.5 | 0 | 2 | 18 | |
| | — | — | — | |
| 0 | 0 | 0 | 24 | |

(8) Triethylene glycol monobutyl ether
First cry assay

| Exposure concentration (mg/L) | \textcircled{1} | \textcircled{2} | \textcircled{3} | EC₅₀ (mg/L) |
|---|---|---|---|---|
| 50 | 0 | 9 | 11 | 57.982 |
| 25 | 0 | 2 | 18 | |
| 12.5 | 0 | 1 | 19 | |
| | — | — | — | |
| 0 | 0 | 0 | 24 | |

(9) Ethylene glycol monophenyl ether
First cry assay

| Exposure concentration (mg/L) | \textcircled{1} | \textcircled{2} | \textcircled{3} | EC₅₀ (mg/L) |
|---|---|---|---|---|
| 100 | 0 | 18 | 2 | 28.083 |
| 50 | 0 | 10 | 10 | |
| 25 | 1 | 10 | 9 | |
| | — | — | — | |
| 0 | 0 | 4 | 20 | |

(10) Nicotinamide
First cry assay

| Exposure concentration (mg/L) | \textcircled{1} | \textcircled{2} | \textcircled{3} | EC₅₀ (mg/L) |
|---|---|---|---|---|
| 100 | 0 | 9 | 11 | >100 |
| 50 | 0 | 2 | 18 | |
| 25 | 0 | 1 | 19 | |
| | — | — | — | |
| 0 | 1 | 5 | 18 | |

(11) Catechol
First cry assay

| Exposure concentration (mg/L) | \textcircled{1} | \textcircled{2} | \textcircled{3} | EC₅₀ (mg/L) |
|---|---|---|---|---|
| 12.5 | 0 | 18 | 2 | 7.385 |
| 6.25 | 1 | 5 | 14 | |
| 3.125 | 0 | 1 | 19 | |
| | — | — | — | |
| 0 | 0 | 0 | 24 | |

(12) Diethylene glycol
First cry assay

| Exposure concentration (mg/L) | \textcircled{1} | \textcircled{2} | \textcircled{3} | EC₅₀ (mg/L) |
|---|---|---|---|---|
| 100 | 0 | 13 | 7 | 86.148 |
| 50 | 1 | 1 | 18 | |
| 25 | 0 | 2 | 18 | |
| | — | — | — | |
| 0 | 0 | 3 | 21 | |

(13) Diethylene glycol monobutyl ether acetate
First cry assay

| Exposure concentration (mg/L) | \textcircled{1} | \textcircled{2} | \textcircled{3} | EC₅₀ (mg/L) |
|---|---|---|---|---|
| 25 | 1 | 19 | 0 | 7.808 |
| 12.5 | 0 | 14 | 6 | |
| 6.25 | 0 | 8 | 12 | |
| | — | — | — | |
| 0 | 0 | 4 | 20 | |

(14) Triethylene glycol diacetate
First cry assay

| Exposure concentration (mg/L) | \textcircled{1} | \textcircled{2} | \textcircled{3} | EC₅₀ (mg/L) |
|---|---|---|---|---|
| 100 | 0 | 12 | 8 | 79.650 |
| 50 | 0 | 6 | 14 | |
| 25 | 0 | 4 | 16 | |
| | — | — | — | |
| 0 | 0 | 0 | 24 | |

(15) Caffeine
First cry assay

| Exposure concentration (mg/L) | \textcircled{1} | \textcircled{2} | \textcircled{3} | EC₅₀ (mg/L) |
|---|---|---|---|---|
| 25 | 0 | 17 | 3 | 8.224 |
| 12.5 | 0 | 13 | 7 | |
| 6.25 | 0 | 8 | 12 | |
| | — | — | — | |
| 0 | 0 | 0 | 24 | |

(16) Tetraethylene glycol monobutyl ether
First cry assay

| Exposure concentration (mg/L) | \textcircled{1} | \textcircled{2} | \textcircled{3} | EC₅₀ (mg/L) |
|---|---|---|---|---|
| 100 | 0 | 18 | 2 | 50.113 |
| 50 | 1 | 7 | 12 | |
| 25 | 0 | 3 | 13 | |
| 12.5 | 0 | 1 | 19 | |
| 0 | 0 | 3 | 21 | |

(17) Ethanol
First cry assay

| Exposure concentration (mg/L) | \textcircled{1} | \textcircled{2} | \textcircled{3} | EC₅₀ (mg/L) |
|---|---|---|---|---|
| 50 | 1 | 10 | 9 | 56.968 |
| 25 | 1 | 5 | 14 | |
| 12.5 | 0 | 4 | 16 | |
| 6.25 | 0 | 5 | 15 | |
| 0 | 0 | 3 | 21 | |

(18) Phenol
First cry assay

| Exposure concentration (mg/L) | \textcircled{1} | \textcircled{2} | \textcircled{3} | EC₅₀ (mg/L) |
|---|---|---|---|---|
| 25 | 1 | 15 | 3 | 8.643 |
| 12.5 | 0 | 14 | 6 | |
| 6.25 | 0 | 7 | 13 | |
| | — | — | — | |
| 0 | 0 | 0 | 24 | |

(19) Tetrachloroethylene
First cry assay

| Exposure concentration (mg/L) | \textcircled{1} | \textcircled{2} | \textcircled{3} | EC₅₀ (mg/L) |
|---|---|---|---|---|
| 50 | 0 | 4 | 16 | >50 |
| 25 | 0 | 4 | 16 | |
| 12.5 | 0 | 2 | 18 | |
| | — | — | — | |
| 0 | 0 | 0 | 24 | |

(20) p-tert-Butylphenol
First cry assay

| Exposure concentration (mg/L) | \textcircled{1} | \textcircled{2} | \textcircled{3} | EC₅₀ (mg/L) |
|---|---|---|---|---|
| 3.125 | 4 | 16 | 0 | 0.980 |
| 1.5625 | 1 | 11 | 8 | |
| 0.78125 | 0 | 9 | 11 | |
| | — | — | — | |
| 0 | 0 | 0 | 24 | |

(21) Nitrobenzene
First cry assay

| Exposure concentration (mg/L) | \textcircled{1} | \textcircled{2} | \textcircled{3} | EC₅₀ (mg/L) |
|---|---|---|---|---|
| 50 | 6 | 6 | 14 | 43.729 |
| 25 | 0 | 4 | 16 | |
| 12.5 | 0 | 3 | 17 | |
| | — | — | — | |
| 0 | 0 | 0 | 24 | |

(22) Isopropyl alcohol
First cry assay

| Exposure concentration (mg/L) | \textcircled{1} | \textcircled{2} | \textcircled{3} | EC₅₀ (mg/L) |
|---|---|---|---|---|
| 50 | 0 | 8 | 12 | >50 |
| 25 | 0 | 4 | 16 | |
| 12.5 | 0 | 1 | 19 | |
| | — | — | — | |
| 0 | 0 | 3 | 21 | |

(23) 1,1,1-Trichloroethane
First cry assay

| Exposure concentration (mg/L) | \textcircled{1} | \textcircled{2} | \textcircled{3} | EC₅₀ (ug/mL) |
|---|---|---|---|---|
| 100 | 0 | 5 | 15 | >100 |
| 50 | 0 | 6 | 14 | |
| 25 | 0 | 2 | 18 | |
| 12.5 | 0 | 4 | 16 | |
| 0 | 0 | 4 | 20 | |

(24) Trichloroethylene
First cry assay

| Exposure concentration (mg/L) | \textcircled{1} | \textcircled{2} | \textcircled{3} | EC₅₀ (mg/L) |
|---|---|---|---|---|
| 100 | 0 | 10 | 10 | 82.005 |
| 50 | 0 | 9 | 11 | |
| 25 | 0 | 3 | 17 | |
| 12.5 | 0 | 1 | 19 | |
| 0 | 0 | 4 | 20 | |

(25) 1,2-Dichloroethane
First cry assay

| Exposure concentration (mg/L) | \textcircled{1} | \textcircled{2} | \textcircled{3} | EC₅₀ (mg/L) |
|---|---|---|---|---|
| 100 | 0 | 9 | 11 | >100 |
| 50 | 0 | 5 | 15 | |
| 25 | 0 | 1 | 19 | |
| | — | — | — | |
| 0 | 1 | 5 | 18 | |

(26) t-butyl hydroquinone
First cry assay

| Exposure concentration (mg/L) | \textcircled{1} | \textcircled{2} | \textcircled{3} | EC₅₀ (mg/L) |
|---|---|---|---|---|
| 6.25 | 9 | 11 | 0 | 1.396 |
| 3.125 | 2 | 18 | 0 | |
| 1.5625 | 0 | 14 | 6 | |
| | — | — | — | |
| 0 | 0 | 0 | 24 | |

(27) Diethylenetriaminepentaacetic acid
First cry assay

| Exposure concentration (mg/L) | \textcircled{1} | \textcircled{2} | \textcircled{3} | EC₅₀ (mg/L) |
|---|---|---|---|---|
| 25 | 2 | 15 | 3 | 9.679 |
| 12.5 | 0 | 12 | 8 | |
| 6.25 | 0 | 9 | 11 | |
| 3.125 | 0 | 0 | 20 | |
| 0 | 0 | 3 | 21 | |

(28) Ethylenediaminetetraacetic acid
First cry assay

| Exposure concentration (mg/L) | \textcircled{1} | \textcircled{2} | \textcircled{3} | EC₅₀ (mg/L) |
|---|---|---|---|---|
| 25 | 0 | 15 | 5 | 16.303 |
| 12.5 | 1 | 7 | 12 | |
| 6.25 | 0 | 1 | 19 | |
| | — | — | — | |
| 0 | 0 | 3 | 21 | |

(29) Nickel (II) chloride
First cry assay

| Exposure concentration (mg/L) | \textcircled{1} | \textcircled{2} | \textcircled{3} | EC₅₀ (mg/L) |
|---|---|---|---|---|
| 50 | 0 | 20 | 0 | 4.243 |
| 25 | 0 | 18 | 2 | |
| 12.5 | 0 | 16 | 4 | |
| 6.25 | 0 | 13 | 7 | |
| 0 | 0 | 3 | 21 | |

(30) 9-cis-Retinoic acid
First cry assay

| Exposure concentration (mg/L) | \textcircled{1} | \textcircled{2} | \textcircled{3} | EC₅₀ (mg/L) |
|---|---|---|---|---|
| 0.09765625 mg/L | 0 | 10 | 10 | 0.086 |
| 0.048828125 mg/L | 0 | 7 | 13 | |
| 0.0244140625 mg/L | 0 | 0 | 20 | |
| | — | — | — | |
| 0 | 0 | 6 | 18 | |

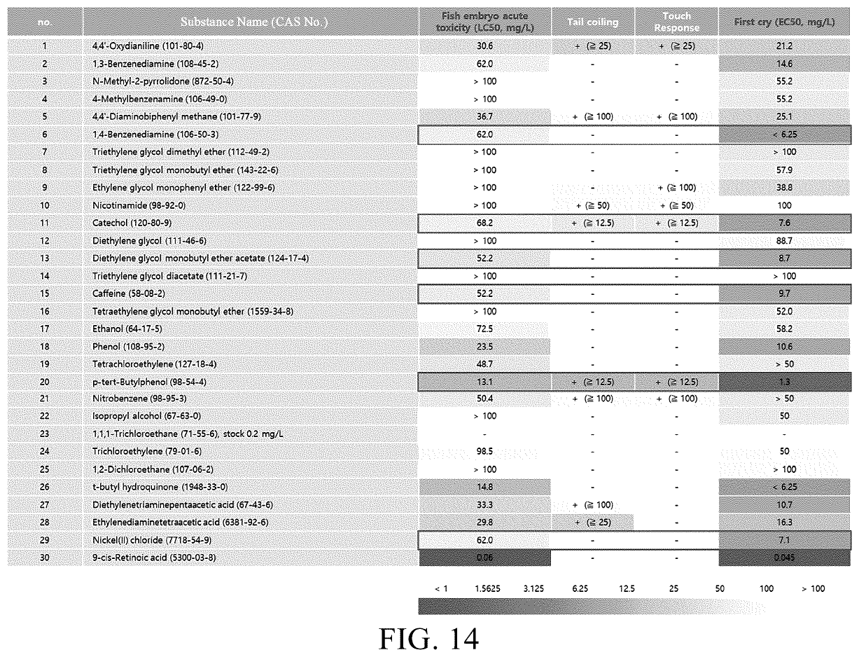

The analysis results for 30 chemical substances were compared with the results of the fish acute toxicity test method (FET test) of OECD and presented in Table 2 and FIG. 14 below. As a result, 24 substances (Nos. 1, 2, 3, 4, 5, 6, 8, 9, 11, 12, 13, 14, 15, 16, 17, 18, 20, 21, 24, 26, 27, 28, 29, and 30 in Table 2 below) showed toxic effects on the swim bladder formation process. In particular, Nos. 6, 11, 13, 15, 20, and 29 in Table 2 showed EC₅₀ values that were up to 10 times lower than the LC₅₀ values obtained using the fish acute toxicity test method (FET) (Table 2 and FIG. 14). This demonstrates that the disclosed alternative method which evaluates swim bladder development is approximately 10 times more sensitive and efficient than the conventional fish acute toxicity test method in identifying developmental neurotoxicity.

TABLE 2

| | | | | Swim Bladder |
|---|---|---|---|---|
| | | | Fish Acute | Development Effect |
| | | | Toxicity | Alternative Test Method |
| | | | Test Method | (First Cry Assay) |
| No. | Substance Name | CAS No. | (LC₅₀, mg/L) | (EC₅₀, mg/L) |
| 1 | 4,4′-Oxydianiline | 101-80-4 | 30.6 | 21.2 |
| 2 | 1,3-Benzenediamine | 108-45-2 | 62.0 | 14.6 |
| 3 | N-Methyl-2-pyrrolidone | 872-50-4 | >100 | 55.2 |
| 4 | 4-Methylbenzenamine | 106-49-0 | >100 | 55.2 |
| 5 | 4,4′-Diaminobiphenyl | 101-77-9 | 36.7 | 25.1 |
| | methane | | | |
| 6 | 1,4-Benzenediamine | 106-50-3 | 62.0 | <6.25 |
| 7 | Triethylene glycol | 112-49-2 | >100 | >100 |
| | dimethyl ether | | | |
| 8 | Triethylene glycol | 143-22-6 | >100 | 57.9 |
| | monobutyl ether | | | |
| 9 | Ethylene glycol | 122-99-6 | >100 | 38.8 |
| | monophenyl ether | | | |
| 10 | Nicotinamide | 98-92-0 | >100 | 100 |
| 11 | Catechol | 120-80-9 | 68.2 | 7.6 |
| 12 | Diethylene glycol | 111-46-6 | >100 | 88.7 |
| 13 | Diethylene glycol | 124-17-4 | 52.2 | 8.7 |
| | monobutyl ether | | | |
| | acetate | | | |
| 14 | Triethylene glycol | 111-21-7 | >100 | >100 |
| | diacetate | | | |
| 15 | Caffeine | 58-08-2 | 52.2 | 9.7 |
| 16 | Tetraethylene glycol | 1559-34-8 | >100 | 52.0 |
| | monobutyl ether | | | |
| 17 | Ethanol | 64-17-5 | 72.5 | 58.2 |
| 18 | Phenol | 108-95-2 | 23.5 | 10.6 |
| 19 | Tetrachloroethylene | 127-18-4 | 48.7 | >50 |
| 20 | p-tert-Butylphenol | 98-54-4 | 13.1 | 1.3 |
| 21 | Nitrobenzene | 98-95-3 | 50.4 | >50 |
| 22 | Isopropyl alcohol | 67-63-0 | >100 | 50 |
| 23 | 1,1,1-Trichloroethane | 71-55-6 | — | — |
| 24 | Trichloroethylene | 79-01-6 | 98.5 | 50 |
| 25 | 1,2-Dichloroethane | 107-06-2 | >100 | >100 |
| 26 | t-butyl hydroquinone | 1948-33-0 | 14.8 | <6.25 |
| 27 | Diethylenetriaminepentaacetic | 67-43-6 | 33.3 | 10.7 |
| | acid | | | |
| 28 | Ethylenediaminetetraacetic | 6381-92-6 | 29.8 | 16.3 |
| | acid | | | |
| 29 | Nickel (II) chloride | 7718-54-9 | 62.0 | 7.1 |
| 30 | 9-cis-Retinoic acid | 5300-03-8 | 0.06 | 0.045 |

While the embodiments have been described with reference to the limited drawings, it will be apparent to one of ordinary skill in the art that various alterations and modifications can be made from the above description. For example, suitable results may be achieved if the described techniques are performed in a different order, and/or if components in a described system, structure, or device are combined in a different manner, and/or replaced or supplemented by other components or their equivalents. Therefore, other implementations, other example embodiments, other aspects, and/or equivalents of the claims are within the scope of the following claims.

The present disclosure provides the following aspects:

Aspect 1. A plate for evaluating toxicants using fish embryos, the plate comprising:
n outer wells; and n inner wells respectively located inside the outer wells, wherein n is an integer of 1≤n≤96,
wherein the height of the inner wells is less than the height of the outer wells.

Aspect 2. The plate of aspect 1, wherein the inner wells are non-permeable.

Aspect 3. The plate of any one of the preceding aspects, wherein the outer wells and the inner wells of the plate are filled with a liquid selected from water and a test solution, and the height of the liquid is less than the height of the outer wells and greater than the height of the inner wells.

Aspect 4. The plate of any one of the preceding aspects, wherein the height of the inner wells is about 5 mm to about 21 mm, and a difference (Δh) between the height of the inner wells and the height of the liquid satisfies the relationship of Equation 1.

$$3 mm \leq Liquid height - Inner well height ( Δ h ) \leq 10 mm \qquad [ Equation 1 ]$$

Aspect 5. The plate of any one of the preceding aspects, wherein the height ratio of the inner wells and the outer wells is about 1:3 to about 1:1.2.

Aspect 6. The plate of any one of the preceding aspects, wherein the shape of each of the outer wells or the inner wells is either a prism or a cylinder.

Aspect 7. The plate of any one of the preceding aspects, wherein the diameter of the outer wells is about 10 mm to about 40 mm and satisfies the relationship of Equation 2.

$$1.1 \leq Outer well diameter / Inner well diameter \leq 4 \qquad [ Equation 2 ]$$

Aspect 8. The plate of any one of the preceding aspects, wherein the fish embryos are zebrafish embryos.

Aspect 9. The plate of any one of the preceding aspects, wherein the plate is for screening developmental neurotoxicants using fish embryos.

Aspect 10. A method of screening developmental neurotoxicants using fish embryos, the method including:
a) providing the plate of any one of aspects 1 to 9;
b) filling the plate with water or a test solution such that the height of the water or the test solution is greater than the height of inner wells and less than the height of outer wells, and positioning fish embryos in the inner wells;
c) when the plate is filled with water in b), introducing a sample into the inner wells of the plate; and
d) identifying whether larvae formed from the fish embryos move between the inner wells and the corresponding outer wells.

Aspect 11. The method of aspect 10, wherein the fish embryos are zebrafish embryos.

Aspect 12. The method of aspect 10, wherein the fish embryos in b) are fish embryos from immediately after fertilization to about 48 hours.

Aspect 13. The method of aspect 10, wherein d) is performed 100 to 150 hours after fertilization of the fish embryos.

Aspect 14. The method of aspect 10, wherein d) includes counting the number of larvae that failed to move between the inner wells and the corresponding outer wells, and the number of larvae that succeeded in moving between the inner wells and the corresponding outer wells, respectively.

Aspect 15. The method of any one of aspects 10 to 14, wherein, among the larvae that succeeded in moving between the inner wells and the corresponding outer wells, the larvae are determined to have succeeded in swim bladder inflation when the swim bladders of the larvae expand into a round, balloon-like shape.

Aspect 16. The method of any one of aspects 10 to 15, further including: e) determining that the sample is a toxicant if the number of larvae that succeeded in swim bladder inflation is 50% or less of the total number of embryos.

Aspect 17. An apparatus for evaluating toxicants using fish embryos, the apparatus including:
the plate of any one of aspects 1 to 9; and
an image reader configured to perform the counting of aspect 14 through automated image analysis.

Aspect 18. The apparatus of aspect 17, wherein the fish embryos are zebrafish embryos.

Aspect 19. A kit for evaluating developmental neurotoxicants using fish embryos, the kit including:
the plate of any one of aspects 1 to 9, and
instructions for using the plate in a method of screening developmental neurotoxicants using fish embryos.

Aspect 20. The kit of aspect 19, wherein the fish embryos are zebrafish embryos.

What is claimed is:

1. A plate for evaluating toxicants using fish embryos, the plate comprising:
n outer wells; and
n inner wells respectively located inside the outer wells, wherein n is an integer of 1≤n≤96,
wherein a height of the inner wells is less than a height of the outer wells.

2. The plate of claim 1, wherein the inner wells are non-permeable.

3. The plate of claim 1, wherein the outer wells and the inner wells of the plate are filled with a liquid selected from water and a test solution, and a height of the liquid is less than the height of the outer wells and greater than the height of the inner wells.

4. The plate of claim 3, wherein the height of the inner wells is about 5 mm to about 21 mm, and a difference (Δh) between the height of the inner wells and the height of the liquid satisfies a relationship of Equation 1.
$$3 mm \leq Liquid height - Inner well height ( Δ h ) \leq 10 mm \qquad [ Equation 1 ]$$

5. The plate of claim 1, wherein a height ratio of the inner wells and the outer wells is about 1:3 to about 1:1.2.

6. The plate of claim 1, wherein a shape of each of the outer wells or the inner wells is either a prism or a cylinder.

7. The plate of claim 1, wherein a diameter of the outer wells is about 10 mm to about 40 mm and satisfies a relationship of Equation 2.
$$1.1 \leq Outer well diameter / Inner well diameter \leq 4 \qquad [ Equation 2 ]$$

8. The plate of claim 1, wherein the fish embryos are zebrafish embryos.

9. The plate of claim 1, wherein the plate is for screening developmental neurotoxicants using fish embryos.

10. A method of screening developmental neurotoxicants using fish embryos, the method comprising:
a) providing the plate of claim 1;
b) filling the plate with water or a test solution such that a height of the water or the test solution is greater than a height of inner wells and less than a height of outer wells, and positioning fish embryos in the inner wells;
c) when the plate is filled with water in b), introducing a sample into the inner wells of the plate; and
d) identifying whether larvae formed from the fish embryos move between the inner wells and the corresponding outer wells.

11. The method of claim 10, wherein the fish embryos are zebrafish embryos.

12. The method of claim 10, wherein the fish embryos in b) are fish embryos from immediately after fertilization to about 48 hours.

13. The method of claim 10, wherein d) is performed 100 to 150 hours after fertilization of the fish embryos.

14. The method of claim 10, wherein d) comprises counting the number of larvae that failed to move between the inner wells and the corresponding outer wells, and the number of larvae that succeeded in moving between the inner wells and the corresponding outer wells, respectively.

15. The method of claim 14, wherein, among the larvae that succeeded in moving between the inner wells and the corresponding outer wells, the larvae are determined to have succeeded in swim bladder inflation when the swim bladders of the larvae expand into a round, balloon-like shape.

16. The method of claim 10, further comprising:
e) determining that the sample is a toxicant if the number of larvae that succeeded in swim bladder inflation is 50% or less of the total number of embryos.

17. An apparatus for evaluating toxicants using fish embryos, the apparatus comprising:
the plate of claim 1; and
an image reader configured to perform the counting of claim 14 through automated image analysis.

18. The apparatus of claim 17, wherein the fish embryos are zebrafish embryos.

19. A kit for evaluating developmental neurotoxicants using fish embryos, the kit comprising:
the plate of claim 1; and
instructions for using the plate of claim 1 in a method of screening developmental neurotoxicants using fish embryos.

20. The kit of claim 19, wherein the fish embryos are zebrafish embryos.
